(19) United States
(12) Patent Application Publication
(10) Pub. No.: US 2025/0331994 A1
(43) Pub. Date: Oct. 30, 2025

(54) SPINAL IMPLANT

(71) Applicant: Mullin Patents, LLC, New Albany, OH (US)

(72) Inventor: Bradford MULLIN, New Albany, OH (US)

(21) Appl. No.: 19/191,096

(22) Filed: Apr. 28, 2025

Related U.S. Application Data
(60) Provisional application No. 63/640,448, filed on Apr. 30, 2024.

(51) Int. Cl. A61F 2/44; A61F 2/30; A61L 27/54; A61L 27/56

(52) U.S. Cl. CPC A61F 2/4455; A61F 2/30749; A61F 2/30771; A61L 27/54; A61L 27/56; A61F 2002/30166; A61F 2002/30772; A61F 2002/30841

(57) ABSTRACT

Presently disclosed is a spinal implant. In an embodiment, a spinal implant is formed of porous titanium and is configured to promote bone growth. The spinal implant may have a first portion configured to be secured by, but not attached to a fixation system attached to one or more vertebra of a spine; and a fusion plate configured to fuse unilateral transverse processes, lamina, or facet of adjacent vertebrae longitudinally along the spine, the fusion plate extending from the first portion and offset from the first portion, such that, when the first portion is secured by the fixation system, the fusion plate is maintained in compression against the transverse processes, lamina, or facet to promote bone growth. A method of fusing adjacent vertebrae of a spine using a spinal implant is also disclosed.

### CROSS-REFERENCE TO RELATED APPLICATION

This application claims prior to and the benefit of U.S. Provisional Patent Application No. 63/640,448, field Apr. 30, 2024, titled SPINAL IMPLANT, the disclosure of which is incorporated herein by reference in its entirety.

### BACKGROUND OF THE INVENTION

The present disclosure relates to a spinal implant, and more particularly, to a posterolateral, laminar and facet fusion device.

Spinal fusion has been developed to immobilize joints as a treatment for various conditions and disorders. Prior interbody implants placed between vertebra have shown improved rates of fusion due to the implant being under compression. Attempts to achieve bone growth in posterolateral vertebra fusion have been less successful than interbody fusion.

Spinal fusions of the lumbar spine typically are located either between the vertebral bodies or in the posterior lateral space. Posterior lateral fusions have the advantage of taking a shorter amount of time, creating less blood loss, and avoiding nerve retraction. When the lamina are not removed, a laminar fusion may be applied. However, fusion rates are lower than interbody fusions. This is thought to be in part because Wolf's law (fusion under compressive forces) cannot be applied. Unless a solid fusion can be obtained, screw and rod fusion constructs will likely break because of metal fatigue. Fusions and screw/rod fracture happen over a variable length of time. Posterior lateral fusions require a source of bone. Traditionally, this can either involve local bone, iliac crest graft, and/or banked bone. Other materials can be applied such as demineralized bone matrix, and synthetic materials. There are variable fusion rates with different types of materials. It is recognized that interbody fusions have the highest fusion rates, followed by posterolateral fusions. This is thought to be because of the application of compressive force.

Posterolateral fusions that require attaching an implant to a rod or bone screw are known, such as in U.S. Pat. Nos. 10,881,525 and 11,737,887 to Dr. Brad Mullin, the disclosures of which are incorporated here by reference. The implant disclosed in those patents must be attached to a rod or bone screw, or similar component of a fixation device. While effective, it has been desired to reduce the complexity of the installation procedure therefore improvements to implants for posterolateral fusion are still needed.

Recently porous titanium has been introduced into fusion devices. To date, it has been used in interbody devices to augment interbody fusions. However, it has not been applied to laminar or posterolateral fusion or facet fusions.

Accordingly, there remains a need for improved spinal implants for posterolateral, laminar and facet fusion that overcome the challenges of these prior solutions.

### SUMMARY OF THE INVENTION

Presently disclosed is a spinal implant. In an embodiment, a spinal implant includes a first portion configured to be secured by, but not attached to, a fixation system attached to one or more vertebra of a spine. The spinal implant also includes a fusion plate configured to fuse unilateral transverse processes, lamina, or facet of adjacent vertebrae longitudinally along the spine, the fusion plate extending from the first portion and offset from the first portion, such that, when the first portion is secured by the fixation system, the fusion plate is maintained in compression against the transverse processes, lamina, or facet to promote bone growth.

In some embodiments, the spinal implant is formed of a porous material selected to promote bone growth. The porous material may be porous titanium. The spinal implant may be a monolithic structure. The fusion plate of the spinal implant may have a convex lower surface, and may have a concave upper surface. The lower surface may include a plurality of protrusions, which may be configured to promote contact with the transverse processes, the lamina, or the facet of the adjacent vertebrae. The upper surface may be configured to receive bone material, and may define a trough.

In some embodiments, the fusion plate extends on opposite sides of the first portion, and may have a generally H-shaped cross section. In embodiments, the fixation system includes a pair of bone fasteners attachable to adjacent vertebrae and a rod extending between the pair of bone fasteners. In some embodiments, the fixation system includes at least one bone fastener having a screw and a tulip, and the spinal implant is configured to be secured in compression between the tulip and the spine.

Also disclosed is an implant system for fusing adjacent vertebrae. In embodiments, the implant system includes means for securing adjacent vertebrae together and thereby inhibiting relative movement of the adjacent vertebrae; means for contacting transverse processes, lamina, or facet of the adjacent vertebrae and promoting bone growth to achieve fusion between the transverse processes, lamina, or facet of the adjacent vertebrae; where the contacting means are not attached to the securing means, but are secured by the securing means to maintain the contacting means in compression with the transverse processes, lamina, or facet to be fused.

Also disclosed is a method of fusing adjacent vertebrae of a spine using a spinal implant. In embodiments, the method comprises the steps of: installing a fixation system for securing adjacent vertebrae, the fixation system including a rod securable to a pair of bone fasteners by a pair of screws; partially tightening the screws such that a distance between the rod and spine is greater than a thickness of the spinal implant; inserting the spinal implant between the rod and the spine without securing the spinal implant to the rod, wherein the spinal implant is configured to fuse unilateral transverse processes, lamina, or facet of adjacent vertebrae longitudinally along the spine; and tightening the screws such that when the rod is secured to the bone fasteners, the spinal implant is maintained in compression against the transverse processes, lamina, or facet to promote bone growth.

Also disclosed is a method of fusing adjacent vertebrae of a spine using a spinal implant, in which the method includes the steps of installing a fixation system for securing adjacent vertebrae, the fixation system including at least one bone fastener including a screw and a tulip; partially tightening the screw of the bone fastener such that a distance between the tulip and the spine is greater than a thickness of the spinal implant; inserting the spinal implant between the tulip and the spine without securing the spinal implant to the screw or the tulip, wherein the spinal implant is configured to fuse unilateral transverse processes, lamina, or facet of adjacent vertebrae longitudinally along the spine; and tightening the screw such that the spinal implant is maintained in compression by the tulip against the transverse processes, lamina, or facet to promote bone growth. In some embodiments, the fixation system further includes at least a second bone fastener having a screw and a tulip, and the method includes securing the spinal implant under the tulip of each bone fastener.

### BRIEF DESCRIPTION OF THE FIGURES

The invention can be understood from the following detailed description of exemplary embodiments of the invention taken in conjunction with the accompanying drawings.

FIG. 1 is a top view of a prior art spinal implant system.

FIG. 2 is bottom view of another embodiment of a prior art spinal implant.

FIG. 3 illustrates a fixation system according to the present disclosure.

FIG. 4 illustrates another view of a fixation system according to the present disclosure.

FIG. 5 illustrates a spinal implant according to the present disclosure.

FIG. 6 illustrates an installed spinal implant according to the present disclosure.

FIG. 7 illustrates another view of an installed spinal implant according to the present disclosure.

### DETAILED DESCRIPTION

This invention relates in general to a spinal implant system for posterolateral, laminar and facet fusion, and methods of fusing adjacent vertebrae using a spinal implant.

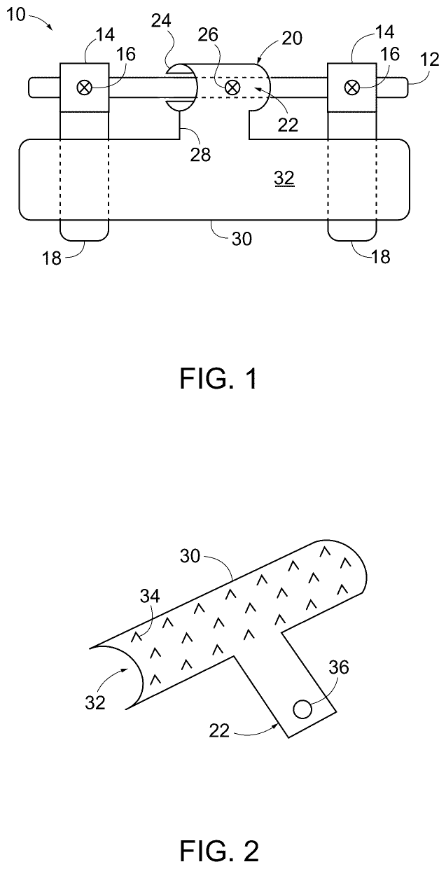

Referring generally to FIGS. 1 and 2, a prior art spinal implant system is disclosed for reference. The spinal implant system 10 includes a fixation system, which includes a rod 12, and bone fasteners 14. As shown in FIG. 1, a pair of bone fasteners 14 are fastened to adjacent vertebra. A rod 12 extends between the pair of bone fasteners 14, and is secured in place by the screws 16. The fixation system thereby inhibits movement of the adjacent vertebra.

The spinal implant system also includes a spinal implant having a body 20. The body 20 may be formed of a porous material selected to promote bone growth. In one embodiment, the body is formed of porous titanium with a modulus similar to nature bone. In other embodiments, selected portions of the body are formed of porous material while other portions are formed of non-porous materials. In some embodiments, the body of the spinal implant is formed by an additive manufacturing process, such as 3D printing. In some embodiments, the porous material forms a lattice having pores of approximately 0.75 millimeter in diameter.

The body 20 generally includes an attachment portion 22 and a fusion plate 30. The attachment portion 20 is configured to secure the spinal implant to a rod 12 of the fixation system. As shown in FIG. 1, the attachment portion 22 includes a hook 24 that extends at least partially around the rod 12. The attachment portion 22 may also include a screw 26. As illustrated, the screw 26 passes through the attachment portion 22 and into a portion of the hook 24 thereby affixing the spinal implant to the rod 12.

In another embodiment, as shown in FIG. 2, the attachment portion 22 includes an aperture 36. The aperture 36 is configured to receive a screw (not shown in FIG. 2) to secure the spinal implant to a bone fastener of the fixation system. In this manner, the spinal implant may be secured to various components of the fixation system to provide the desired alignment with the transverse processes to be fused.

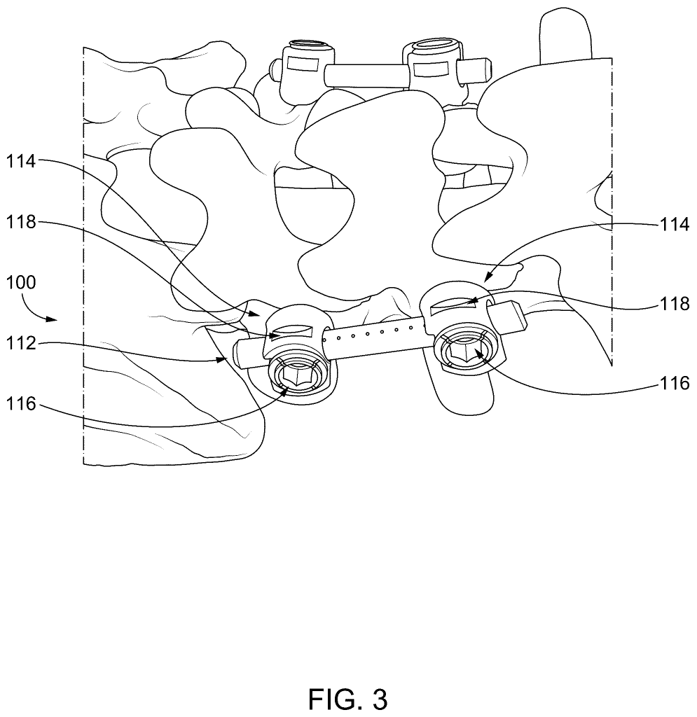

Referring now to FIGS. 3-8, an improved spinal implant system for posterolateral, laminar and facet fusion, and methods of fusing adjacent vertebrae using the improved spinal implant are disclosed.

In various embodiments, the presently disclosed spinal implant includes a first portion configured to be secured by a fixation system attached to one or more vertebra of a spine. In contrast to the prior art spinal implant shown in FIGS. 1-2, the presently disclosed spinal implant is not attached to the fixation system. Because the presently disclosed spinal implant is not attached to the fixation system, use of the spinal implant may be simplified and less invasive to the patient.

The presently disclosed spinal implant also includes a fusion plate configured to fuse unilateral transverse processes, lamina, or facet of adjacent vertebrae longitudinally along the spine. In embodiments, the fusion plate extends from the first portion and is offset from the first portion, such that, when the first portion is secured by the fixation system, the fusion plate is maintained in compression against the transverse processes, lamina, or facet to promote bone growth.

Referring to FIG. 3, a fixation system 100 is illustrated. The fixation system 100 includes a rod 112 and a pair of bone fasteners 114. Each bone fastener 114 includes a bone screw (not visible in FIG. 3) which is inserted into the vertebrae and a tulip 118 attached to the head of the screw. In some embodiments, the tulip 118 is configured to receive the rod 112. As shown in FIG. 3, the pair of bone fasteners 114 are fastened to adjacent vertebra on the same side (unilateral) of the spine. The rod 112 extends between the pair of bone fasteners 114 and is secured in place by set screws 116. The fixation system thereby inhibits movement of the adjacent vertebra. As illustrated, the space between the tulip 118 and the spine is reduced when the bone screw is tightened to secure the bone fastener to the vertebrae. In addition, the space between the rod 112 and the spine is reduced when screws 116 are tightened to secure the rod 112 to the bone fasteners 114.

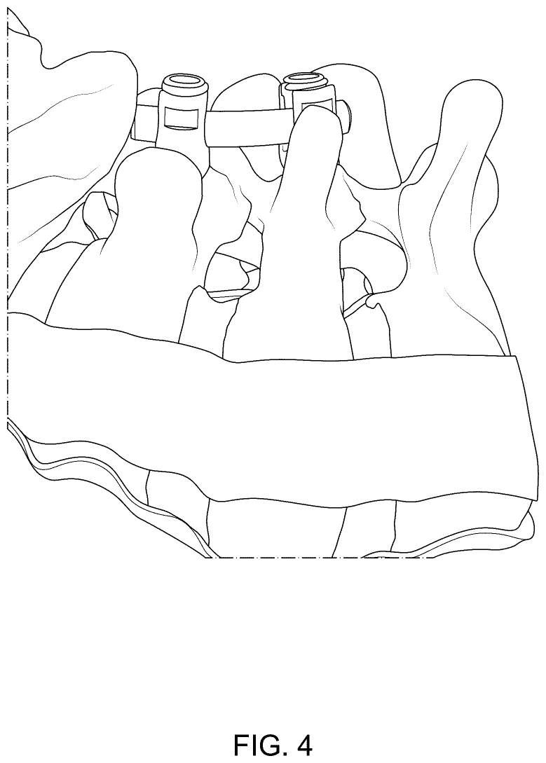

FIG. 4 illustrates another view of the fixation system 100 secured to the spine without the spinal implant.

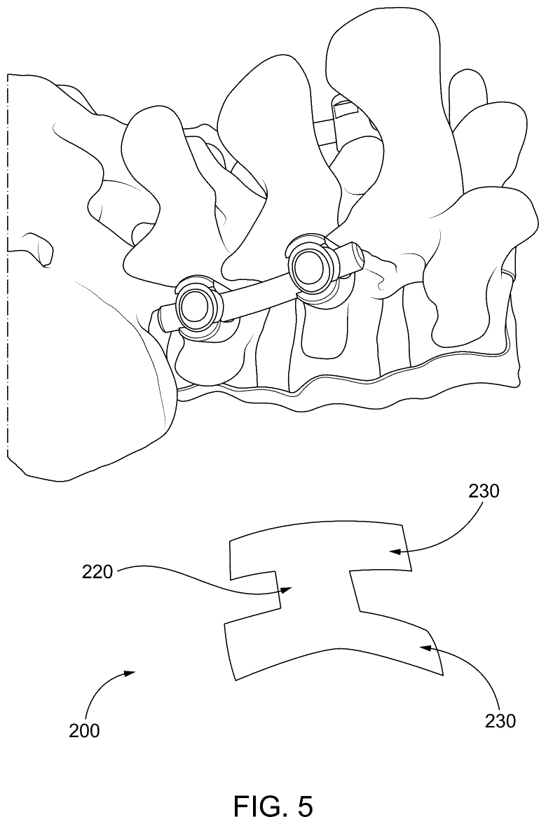

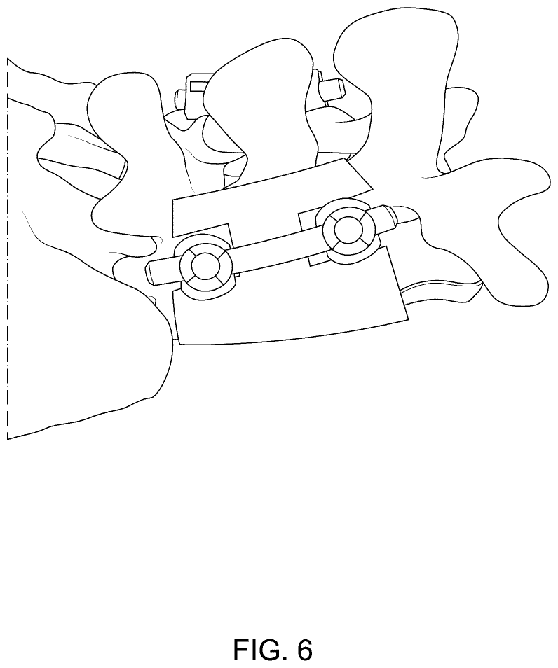

Referring to FIGS. 5 and 6, an embodiment of the presently disclosed spinal implant is illustrated. The spinal implant 200 includes a first portion 220 and a fusion plate 230. The first portion 220 is configured to be placed between a fixation system and the spine. The first portion 220 is not configured to attach to the fixation system; rather, the first portion is held in compression between a component of the fixation system, such as a rod 212 or a tulip 118 of the bone fastener 114, and the spine. In this manner, the spinal implant may be conveniently inserted without additional tools or components other than the fixation system used to immobilize adjacent vertebrae.

The spinal implant 200 also includes a fusion plate 230 that extends from and is integral with the first portion 220. The fusion plate 230 is configured to contact portions of the adjacent vertebrae to be fused, such as the adjacent transverse processes 18. Alternatively, the fusion plate 230 may contact adjacent lamina or facet of the adjacent vertebrae to be fused. In each case, the fusion plate 230 contacts surfaces only on one side of the spine (i.e. unilateral). In one embodiment, the fusion plate 230 may have a generally H-shaped cross-section (when viewed from above in FIG. 5) such that when installed, the fusion plate extends longitudinally along the spine. In other embodiments, the fusion plate may have an oval or rounded profile as desired to accommodate the specific geometry of an individual patient's spine.

The shape of the spinal implant 200 may be modified in various ways such that the first portion 220 is configured to be inserted between the fixation system and the spine and the fusion plate 230 extends on one or both sides of the first portion as desired. The first portion 220 of the spinal implant may also fuse to the portion of the vertebrae.

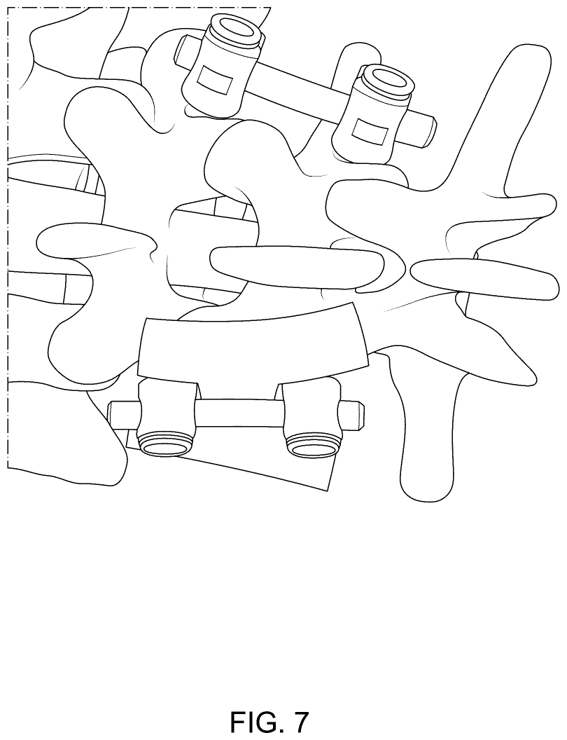

Referring now to FIGS. 6 and 7, the spinal implant 200 is shown in use. As illustrated, the spinal implant 200 is secured between the tulip 118 of the fixation system 100 and the patient's spine. In addition, the spinal implant 200 may be secured between the rod 112 of the fixation system 100 and the patient's spine.

The fusion plate 230 may be further configured to improve contact with the spine and promote bone growth to achieve the desired fusion. The fusion plate 230 may also be configured to contact the lamina or facet as noted above. In some embodiments, the fusion plate 230 has a convex lower surface such as illustrated in FIG. 2. A convex surface may generally conform to the contours of a patient's transverse processes, lamina or facet. To further improve contact, the fusion plate 230 may include a plurality of protrusions (such as protrusions 34 shown in FIG. 2) from the convex lower surface. In some embodiments, the protrusions are localized raised portions of the lower surface that contact the spine. The protrusions may include repeating or random patterns of such raised portions. When the spinal implant is installed, the protrusions contacting the spine apply increased pressure which is expected to further promote bone growth.

In some embodiments, the fusion plate 230 also includes a concave upper surface that forms a trough 32 (as shown in FIG. 2). The trough may be configured to receive bone material, such as bone chips, bone powder, or a slurry. The addition of bone materials may further promote the growth of new bone and accelerate the fusion of the transverse processes with the spinal implant.

In yet another embodiment, the spinal implant 200 is formed an two or more layers such that bone material may be positioned between the layers of the spinal implant. The layers of the spinal implant 200 may be connected or may be separate allowing each layer to be positioned separately. When the spinal implant is secured by the fixation system, all layers of the spinal implant and any bone material provided between the layers are held in compression against the spine.

In one example, when the spinal implant is installed in a patient, in one example, the spinal implant 200 is inserted between the tulip 118 of a bone fastener 114 of the fixation system 100 and the spine. When the bone screw associated with the tulip 118 is tightened, the spinal implant 200 is compressed between the tulip 118 and the spine.

In another example, when the spinal implant is installed in a patient, in one example, the spinal implant 200 is inserted between the rod 112 of the fixation system 100 and the spine. When the screws 116 are tightened, the rod is secured to the bone fasteners 114 and the spinal implant 200 is compressed between the rod 112 and the spine.

By applying pressure at the points of contact, the spinal implant achieves compression that promotes bone growth in a manner not previously possible with prior art posterolateral vertebrae fusion devices. In this manner, the presently disclosed spinal implant may achieve an improved rate of fusion, which may be comparable to the rate of fusion presently available with interbody devices, but without the drawback and limitations inherent in such interbody devices.

In one embodiment, a method of fusing adjacent vertebrae of a spine using a spinal implant 200 is also disclosed. Referring again to FIGS. 3-8, a fixation system 100 is installed for securing the adjacent vertebrae. The fixation system 100 includes at least one bone fastener 114, and each bone fastener 114 includes a bone screw and a tulip 118 attached to the bone screw. Next, the bone screw is partially tightened such that a distance between the tulip 118 and the spine is greater than a thickness of the spinal implant. Partially tightening the bone screws ensures the tulip 118 remains in place while the spinal implant is positioned.

Next, the spinal implant 200 is inserted between the tulip 118 and the spine without securing the spinal implant to the tulip, the bone screw or any other component of the fixation system 100. The spinal implant 200 is configured to fuse the unilateral transverse processes, lamina, or facet of the adjacent vertebrae longitudinally along the spine as described above. Once the spinal implant 200 is positioned between the tulip 118 and the spine, the bone screw is fully tightened to secure the bone fastener 114, such that the spinal implant 200 is maintained in compression against the transverse processes, lamina, or facet to promote bone growth. The method does not require attaching the spinal implant to the fixation system and therefore enables a simplified installation procedure that may be less invasive for the patient.

In another embodiment, a method of fusing adjacent vertebrae of a spine using a spinal implant 200 is also disclosed. Referring again to FIGS. 3-8, a fixation system 100 is installed for securing the adjacent vertebrae. The fixation system 100 includes a rod 112 securable to a pair of bone fasteners 114 by a pair of screws 116. Next, the screws 116 are partially tightened such that a distance between the rod 112 and the spine is greater than a thickness of the spinal implant. Partially tightening the screws ensures the rod 112 remains in place while the spinal implant is positioned.

Next, the spinal implant 200 is inserted between the rod 112 and the spine without securing the spinal implant to the rod. The spinal implant 200 is configured to fuse the unilateral transverse processes, lamina, or facet of the adjacent vertebrae longitudinally along the spine as described above. Once the spinal implant 200 is positioned between the fixation system 100 and the spine, the screws 116 are fully tightened to secure the rod 112 to the bone fasteners 114. When the rod 112 is secured to the bone fasteners 114 the spinal implant 200 is maintained in compression against the transverse processes, lamina, or facet to promote bone growth. The method does not require attaching the spinal implant to the fixation system and therefore enables a simplified installation procedure that may be less invasive for the patient.

A kit of spinal implants may also be provided that includes a selection of spinal implants of different sizes. A surgeon may select the spinal implant best suited to the particular size and geometry of the patient undergoing treatment. In this manner, the presently disclosed spinal implant may be used in treatment of a wide variety of patients.

The presently disclosed spinal implant has been described primary in connection with fusions of the transverse processes, however, as will be understood the spinal implant may also provide for fusions of the lamina or facet.

The presently disclosed spinal implant system may provide numerous advantages for posterolateral, laminar and facet fusion. A spinal implant formed of porous titanium manufactured with an additive manufacturing process may allow bone growth into it and participate in the fusion. Such material is expected to fuse to the transverse processes and can be followed by eventual bony fusion of graft material. Compression can be applied to the spinal implant against the transverse processes. Local bone may be placed between the transverse processes and the spinal implant. This would put compression on either the spinal implant and the transverse process or the local bone. Graft material that is laid to bridge defects would also be placed under compression. The spinal implant would connect to and lock to the fixation system, such as the rod with a set screw. The spinal implant has a pore structure that allows bone growth. Local bone may be trapped under the spinal implant creating a compressed area that would further augment the fusion.

In one embodiment, the presently disclosed spinal implant may be a monolithic structure, formed of porous titanium. The additive manufacturing process described above allows for the first portion and fusion plate of the spinal implant to be formed as an integrated structure avoiding any discontinuities that would impede fusion or bone growth through the implant.

The components of spinal implant 200 can be fabricated from biologically acceptable materials suitable for medical applications, including metals, synthetic polymers, ceramics and bone material and/or their composites. For example, the components of spinal implant 200, individually or collectively, can be fabricated from materials such as stainless steel alloys, commercially pure titanium, titanium alloys, Grade 5 titanium, super elastic titanium alloys, cobalt-chrome alloys, stainless steel alloys, super elastic metallic alloys (e.g., Nitinol, super elasto-plastic metals, such as GUM METAL®, manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO.sub.4 polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate such as hydroxyapatite (HA), corraline HA, biphasic calcium phosphate, tricalcium phosphate, or fluorapatite, tricalcium phosphate (TCP), HA-TCP, calcium sulfate, or other resorbable polymers such as polyaetide, polyglycolide, polytyrosine carbonate, polycaroplaetohe and their combinations, biocompatible ceramics, mineralized collagen, bioactive glasses, porous metals, bone particles, bone fibers, morselized bone chips, bone morphogenetic proteins (BMP), such as BMP-2, BMP-4, BMP-7, rhBMP-2, or rhBMP-7, demineralized bone matrix (DBM), transforming growth factors (TGF, e.g., TGF-(3), osteoblast cells, growth and differentiation factor (GDF), insulin-like growth factor 1, platelet-derived growth factor, fibroblast growth factor, or any combination thereof.

Various components of spinal implant 200 may have material composites, including the above materials, to achieve various desired characteristics such as strength, rigidity, elasticity, compliance, biomechanical performance, durability and radiolucency or imaging preference. The components of spinal implant 200, individually or collectively, may also be fabricated from a heterogeneous material such as a combination of two or more of the above-described materials. The components of spinal implant 200 may be monolithically formed, integrally connected or include fastening elements and/or instruments, as described herein. In one embodiment, a spinal implant, as described herein, may be formed substantially of a biocompatible metal, such as titanium and selectively coated with a bone-growth promoting material, such as HA. In one embodiment, a spinal implant, as described herein, may be formed substantially of a biocompatible polymer, such as PEEK, and selectively coated with a biocompatible metal, such as titanium, or a bone-growth promoting material, such as HA. In some embodiments, titanium may be plasma sprayed onto surfaces of the spinal implant to modify a radiographic signature of the spinal implant and/or improve bony ongrowth to the spinal implant by application of a porous or semi-porous coating of titanium.

While principles and modes of operation have been explained and illustrated with regard to particular embodiments, it must be understood, however, that this may be practiced otherwise than as specifically explained and illustrated without departing from its spirit or scope.

What is claimed is:

1. A spinal implant comprising:
a first portion configured to be secured by, but not attached to, a fixation system attached to one or more vertebra of a spine; and
a fusion plate configured to fuse unilateral transverse processes, lamina, or facet of adjacent vertebrae longitudinally along the spine, the fusion plate extending from the first portion and offset from the first portion, such that, when the first portion is secured by the fixation system, the fusion plate is maintained in compression against the transverse processes, lamina, or facet to promote bone growth.

2. The spinal implant of claim 1, wherein the spinal implant comprises a porous material selected to promote bone growth.

3. The spinal implant of claim 2, wherein the porous material comprises porous titanium.

4. The spinal implant of claim 3, wherein the spinal implant is a monolithic structure.

5. The spinal implant of claim 1, wherein the fusion plate includes a convex lower surface.

6. The spinal implant of claim 5, wherein the fusion plate includes a plurality of protrusions on the convex lower surface.

7. The spinal implant of claim 6, wherein the plurality of protrusions are configured to promote contact with the transverse processes, the lamina, or the facet of the adjacent vertebrae.

8. The spinal implant of claim 1, wherein the fusion plate includes a concave upper surface configured to receive bone material.

9. The spinal implant of claim 8, wherein the concave upper surface defines a trough.

10. The spinal implant of claim 1, wherein the fusion plate extends on opposite sides of the first portion.

11. The spinal implant of claim 10, wherein the spinal implant has an H-shaped cross section.

12. The spinal implant of claim 1, wherein the fixation system includes a pair of bone fasteners attachable to adjacent vertebrae and a rod extending between the pair of bone fasteners.

13. The spinal implant of claim 1, wherein the fixation system includes at least one bone fastener having a screw and a tulip.

14. An implant system for fusing adjacent vertebrae, the implant comprising:
means for securing adjacent vertebrae together and thereby inhibiting relative movement of the adjacent vertebrae;
means for contacting transverse processes, lamina, or facet of the adjacent vertebrae and promoting bone growth to achieve fusion between the transverse processes, lamina, or facet of the adjacent vertebrae;
where the contacting means are not attached to the securing means but are secured by the securing means to maintain the contacting means in compression with the transverse processes, lamina, or facet to be fused.

15. A method of fusing adjacent vertebrae of a spine using a spinal implant, the method comprising:
installing a fixation system for securing adjacent vertebrae, the fixation system including a rod securable to a pair of bone fasteners by a pair of screws;
partially tightening the screws such that a distance between the rod and spine is greater than a thickness of the spinal implant;
inserting the spinal implant between the rod and the spine without securing the spinal implant to the rod, wherein the spinal implant is configured to fuse unilateral transverse processes, lamina, or facet of adjacent vertebrae longitudinally along the spine; and
tightening the screws such that when the rod is secured to the bone fasteners, the spinal implant is maintained in compression against the transverse processes, lamina, or facet to promote bone growth.

16. The method of claim 15, wherein the spinal implant includes:
a first portion configured to be secured by, but not attached to the rod; and
a fusion plate configured to fuse the unilateral transverse processes, lamina, or facet of adjacent vertebrae longitudinally along the spine, the fusion plate extending from one or both sides of the first portion and offset from the first portion, such that, when the first portion is secured by the rod, the fusion plate is maintained in compression against the transverse processes, lamina, or facet to promote bone growth.

17. The method of claim 15, wherein the spinal implant comprises a porous material selected to promote bone growth.

18. The method of claim 17, wherein the porous material comprises porous titanium.

19. The method of claim 15, wherein the spinal implant has an H-shaped cross section and is configured to pass between the rod and the spine when the rod is partially secured to the bone fasteners.

20. A method of fusing adjacent vertebrae of a spine using a spinal implant, the method comprising:
installing a fixation system for securing adjacent vertebrae, the fixation system including at least one bone fastener including a screw and a tulip;
partially tightening the screw of the bone fastener such that a distance between the tulip and the spine is greater than a thickness of the spinal implant;
inserting the spinal implant between the tulip and the spine without securing the spinal implant to the screw or the tulip, wherein the spinal implant is configured to fuse unilateral transverse processes, lamina, or facet of adjacent vertebrae longitudinally along the spine; and
tightening the screw such that the spinal implant is maintained in compression by the tulip against the transverse processes, lamina, or facet to promote bone growth.

21. The method of claim 20, wherein the spinal implant includes:
a first portion configured to be secured by, but not attached to the tulip; and
a fusion plate configured to fuse the unilateral transverse processes, lamina, or facet of adjacent vertebrae longitudinally along the spine, the fusion plate extending from one or both sides of the first portion and offset from the first portion, such that, when the first portion is secured by the tulip, the fusion plate is maintained in compression against the transverse processes, lamina, or facet to promote bone growth.

22. The method of claim 20, wherein the spinal implant comprises a porous material selected to promote bone growth.

23. The method of claim 20, wherein the porous material comprises porous titanium.

24. The method of claim 20, wherein the spinal implant has an H-shaped cross section.

25. The method of claim 20, wherein the fixation system further includes at least a second bone fastener having a screw and a tulip, and the method includes securing the spinal implant under the tulip of each bone fastener.
